(19) United States
(12) Patent Application Publication
(10) Pub. No.: US 2008/0194950 A1
(43) Pub. Date: Aug. 14, 2008

(54) ULTRASOUND IMAGING REMOTE CONTROL UNIT

(75) Inventors: Claudio Patricio Mejia, Wauwatosa, WI (US); Gregory Rachvalsky, Haifa (IL); Scott Raymond Kosloske, Slinger, WI (US)

Correspondence Address: MCANDREWS HELD & MALLOY, LTD, 500 WEST MADISON STREET, SUITE 3400, CHICAGO, IL, 60661, US

(73) Assignee: GENERAL ELECTRIC COMPANY, Schenectady, NY (US)

(21) Appl. No.: 11/674,556

(22) Filed: Feb. 13, 2007

(51) Int. Cl. A61B 8/00

(52) U.S. Cl. 600/437

(57) ABSTRACT

Certain embodiments of the present invention provide methods and systems for operation of an ultrasound system using a remote control. Certain embodiments provide an ultrasound system operable by remote control. The ultrasound system includes an ultrasound scanner configured to obtain image data in at least one ultrasound imaging mode. The system also includes a remote control configured to remotely issue commands for operation of the ultrasound scanner. The remote control is separate from and in communication with the ultrasound scanner. The remote control includes a plurality of controls corresponding to controls of the ultrasound scanner. The remote control also includes a transmitter configured to send/receive one or more commands to/from the ultrasound scanner. Additionally, the remote control includes a processor configured to receive input from the plurality of controls and translate the input to one or more ultrasound scanner commands for transmission to the ultrasound scanner via the transmitter.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to ultrasound system control. In particular, the present invention relates to a remote control for remote operation of an ultrasound system.

A combination of yearly double digit increases in imaging demand and a continuing shortage of technologists and radiologists is resulting in increasing patient imaging exam order error rates. Some estimates place the number of errors as high as 30-40%. Limited Diagnostic Imaging (DI) scanner technologist access to Radiology Information Systems (RIS) and other healthcare information systems allows patient order errors to be propagated throughout a fully or partially digitized healthcare system. Diagnostic Imaging service providers are increasingly compelled to devote one or more FTE's (full time employees) exclusively to quality control to chase down errors and make manual corrections throughout the Diagnostic Imaging service chain.

Additionally, healthcare environments, such as hospitals or clinics, include information systems, such as hospital information systems (HIS), radiology information systems (RIS), clinical information systems (CIS), and cardiovascular information systems (CVIS), and storage systems, such as picture archiving and communication systems (PACS), library information systems (LIS), and electronic medical records (EMR). Information stored may include patient medical histories, imaging data, test results, diagnosis information, management information, and/or scheduling information, for example. The information may be centrally stored or divided at a plurality of locations. Healthcare practitioners may desire to access patient information or other information at various points in a healthcare workflow. For example, during an imaging scan of a patient, medical personnel may access patient information, such as the patient exam order, that are stored in a medical information system. Alternatively, medical personnel may enter new information, such as history, diagnostic, or treatment information, into a medical information system during an imaging scan.

Currently, ultrasound systems can receive waveform data from signal acquisition devices but not in the context of an integrated EP/Hemo and ultrasound environment. Additionally, waveform data has typically been acquired and later provided to an ultrasound system. Furthermore, current systems do not allow real-time or substantially real-time use of physiological waveform data with ultrasound imaging data in an EP/Hemo or ultrasound system.

Currently, multiple personnel may be required to operate an ultrasound system and another system, such as an EP and/or Hemo system, RIS, etc. Users must be at the controls of the system(s) being used in order to operate those systems. There is a need for systems and methods for remote operation of ultrasound and other systems. There is a need for systems and methods allowing a single operator to perform multiple tasks during a procedure. There is a need for systems and methods allowing an operator operate an ultrasound device outside a patient vicinity.

### BRIEF SUMMARY OF THE INVENTION

Certain embodiments of the present invention provide methods and systems for operation of an ultrasound system using a remote control.

Certain embodiments provide an ultrasound system operable by remote control. The ultrasound system includes an ultrasound scanner configured to obtain image data in at least one ultrasound imaging mode. The system also includes a remote control configured to remotely issue commands for operation of the ultrasound scanner. The remote control is separate from and in communication with the ultrasound scanner. The remote control includes a plurality of controls corresponding to controls of the ultrasound scanner. The remote control also includes a transmitter configured to send one or more commands to the ultrasound scanner. Additionally, the remote control includes a processor configured to receive input from the plurality of controls and translate the input to one or more ultrasound scanner commands for transmission to the ultrasound scanner via the transmitter.

Certain embodiments provide a remote control unit configured to remotely issue commands for operation of an ultrasound system. The remote control unit is separate from and in communication with the ultrasound system. The remote control unit includes a plurality of controls corresponding to controls of the ultrasound system. The remote control unit also includes a transmitter configured to send one or more commands to the ultrasound system. The remote control unit further includes a processor configured to receive input from the plurality of controls and translate the input to one or more ultrasound system commands for transmission to the ultrasound system via the transmitter.

Certain embodiments provide a method for remote control of an ultrasound system. The method includes accepting input from at least one of a plurality of controls at an ultrasound system remote control. The method also includes translating the input to one or more ultrasound system commands for transmission to the ultrasound system. The method further includes transmitting the one or more commands from the remote control to an ultrasound system for execution of the one or more commands at the ultrasound system.

### BRIEF DESCRIPTION OF SEVERAL VIEWS OF THE DRAWINGS

FIG. 1 illustrates a high-level system diagram of an EP/Hemo and ultrasound system in accordance with an embodiment of the present invention.

FIG. 2 illustrates a diagram of an EP/Hemo and ultrasound system in accordance with an embodiment of the present invention.

FIG. 3 illustrates a block diagram for a system for interactive communication between a healthcare information system and an imaging system in accordance with an embodiment of the present invention.

FIG. 4 illustrates a remote control for an ultrasound system in accordance with an embodiment of the present invention.

FIG. 5 illustrates a flow diagram for a method for remote control of an ultrasound system in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

In certain embodiments, a plurality of healthcare systems and/or functionality may be combined and/or linked in a variety of combinations. For example, a diagnostic imaging system, a physiological monitoring system and/or information system may be combined and/or operably linked for coordinated operation.

Hemodynamic (hemo) monitoring can aid in detection, identification, and treatment of life-threatening conditions such as heart failure and cardiac tampanade. Using invasive hemodynamic monitoring, for example, a practitioner can help evaluate a patient's response to treatment, such as drugs and mechanical support. A practitioner can evaluate the effectiveness of cardiovascular function such as cardiac output and cardiac index.

Electrophysiological (EP) data includes an analysis of the electrical conduction system of a patient's heart, which generates a heart beat. Catheters may be insert in a vein are then passed into the heart under fluoroscopic guidance, for example. The catheters measure the electrical signals generated by the heart to obtain a more detailed analysis of the electrical signals than a simple electrocardiogram (ECG).

Invasive and/or noninvasive techniques can be used to determine hemodynamic and/or electrophysiological data for a patient. For example, a patient's blood pressure may be measured using a cuff, and/or pressure with a heart may be measured invasively using a catheter. Blood and/or heart pressure measurement may include a systolic pressure and a diastolic pressure. Using the two measurements, a mean pressure can be calculated. Parameters such as chest cardiac output (CO), cardiac index (CI), pulmonary artery wage pressures (PAWP), and cardiac index (CI) may be measuring using a catheter.

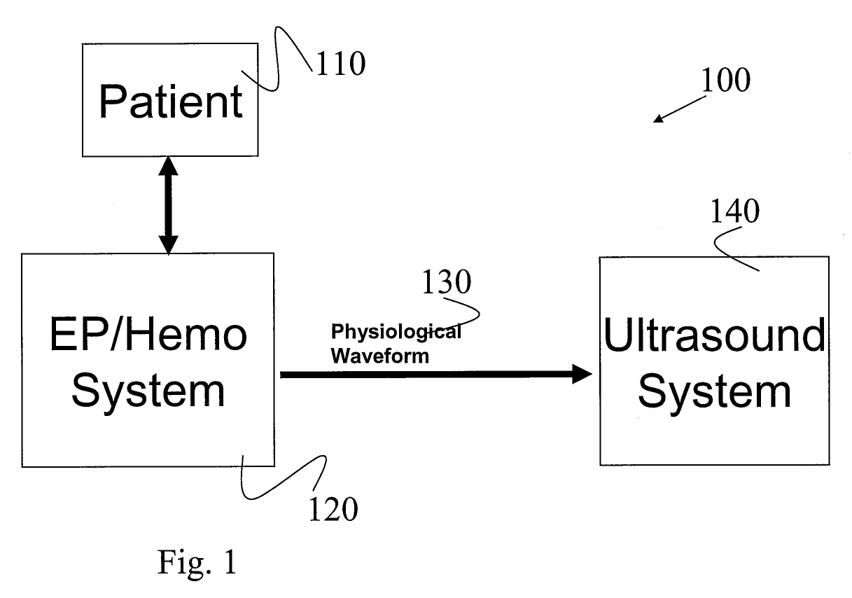

FIG. 1 illustrates a high-level system diagram of an EP/Hemo and ultrasound system 100 in accordance with an embodiment of the present invention. The system 100 includes a patient providing data 110 to an EP/Hemo system 120. The EP/Hemo system 120 sends physiological waveform data 130 and/or other data to an ultrasound system 140 (e.g., a Vivid-I or other ultrasound system). In certain embodiments, the systems 120 and 140 may operate together. In certain embodiments, the systems 120 and 140 may operate separately.

The EP/Hemo system 120 obtains EP and/or hemo data for a patient. In order to share information, the EP/Hemo system 120 may provide one or more interfaces to hospital information systems, database systems and/or other Hemo/EP lab equipment, for example. The information can be collected before, during and/or after a catheterization procedure and may be shared with laboratory and hospital repository systems (e.g., orders and results) for a patient record. Interface(s) may be based on industry-standard protocols (e.g., HL7, SQL, ASCII, DICOM, proprietary protocols, etc.) and/or specific interface(s) for systems that do not support standard protocols, for example. The interface(s) allow exchange and sharing of data (e.g., demographics, history, log, results etc.) between different systems and vendors, for example.

The EP/Hemo system 120 can combine hemodynamic and electrophysiological monitoring into a single system configuration to allow dual use of a catheterization or other lab. EP and hemo data can be stored in a single database to help streamline documentation and access to patient information. The EP/Hemo system 120 provides laboratory performance and resources for patient care. In certain embodiments, the EP/Hemo system 120 may be used in one or more locations, as well as in transit, for example. In certain embodiments, the EP/Hemo system 120 may be accessed remotely. In certain embodiments, the EP/Hemo system 120 may be controlled remotely.

In certain embodiments, the EP/Hemo system 120 includes a graphical user interface to facilitate user-defined procedural lists, macros and configurable electronic documentation. The EP/Hemo system 120 may include a multi-parameter module, such as a GE TRAM® module, that acquires and processes patient physiological parameters, such as ECG, invasive blood pressure, non-invasive blood pressure, pulse oximetry, cardiac output, temperature, respiration, etc. Patient data may be measured in real-time and/or substantially real-time, for example. The EP/Hemo system 120 may also be configured for administrative reporting and facilitation of clinical workflow. The EP/Hemo system 120 may further provide on-line help resources and an ability to save data to a network and/or attached storage, for example.

The EP/Hemo system 120 may include a variety of inputs/outputs, such as one or more ECG leads, one or more stimulation inputs, one or more invasive pressure signals, one or more recording channels, one or more intracardiac channels, one or more catheter inputs, etc. The EP/Hemo system 120 provides diagnostic tools, as well as intracardiac and ECG recording capability, for example. In certain embodiments, the system 120 provides bi-polar channel scalability, automated clinical features and activation mapping to aid in diagnosis. The system 120 may provide a 3D mapping interface as well as connectivity to external system(s), for example. In certain embodiments, the EP/Hemo system 120 may interface uni- or bi-directionally with another system, such as a navigation and/or ablation system to share information, such as mapping events, clinical data and/or EP report data. The EP/Hemo system 120 may be configured to operate in a plurality of languages.

In certain embodiments, the ultrasound system 140 may be configured to provide one or more data acquisition modes and/or data processing capabilities, for example. The system 140 may include one or more probes, such as phased array sector probes, linear array probes, convex array (curved) probes, Doppler pencil probes, multiplane transesophageal phased array probes, etc. The system 140 provides imaging in one or more modes such as 2D mode, M mode, anatomical M mode, color Doppler, color angio, color M mode, anatomical color M mode, spectral Doppler, Pulsed Wave/High Pulse Repetition Frequency (PW/HPRF) Doppler, Tissue Doppler, CW Doppler, etc.

The ultrasound system 140 may be configured for a variety of data processing. The system 140 may provide echo data processing of phase, amplitude and frequency, for example. The system 140 may provide digital raw data replay for image post post-processing and offline measurement and analysis. The system 140 may include an instant review screen to display one or more loops/images for study review. In certain embodiments, a scan plane position indicator and probe temperature may be displayed with multi-plane transesophageal probes. An image orientation indicator may be displayed with image data.

A display integrated with and/or associated with the ultrasound system 140 may be configured for a plurality of views including single, dual and quad-screen view. In certain embodiments, the system 140 provides a selectable display configuration of duplex and triplex modes either side-by-side or top-bottom.

In certain embodiments the ultrasound system 140 provides variable transmit frequencies for resolution/penetration improvement. The system 140 may also provide variable contour filtering for edge enhancement.

The ultrasound system 140 may also provide a variety of analysis and workflow tools. For example, personalized measurement protocols allow individual setting and ordering of measurement and analysis items. Measurements may be labeled using protocols and/or post assignments, for example. Bodymark icons may be provided for location and position of a probe. In certain embodiments, the system 140 provides cardiac calculation and/or vascular measurement functionality including measurement and display of multiple, repeated measurements. In certain embodiments, measurements are assignable to one or more protocols and/or report generators. Parameter(s) and/or parameter annotation may follow a medical standard, such as an American Society of Echocardiography standard, and/or may be user-assignable, for example. Certain embodiments provide a Doppler auto-trace function including automatic calculation in live and/or digital replay, for example. Functions, such as data storage and report creation, may be combined and/or automated in a variety of ways, for example.

In certain embodiments, the ultrasound system 140 and/or the EP/Hemo system 120 may access a knowledge database and/or guidance center, such as GE's iLinq™ system, for system-specific and/or context-sensitive support. The system 120 and/or system 140 may also communicate with a remote diagnostic and support center, such as GE's InSite™.

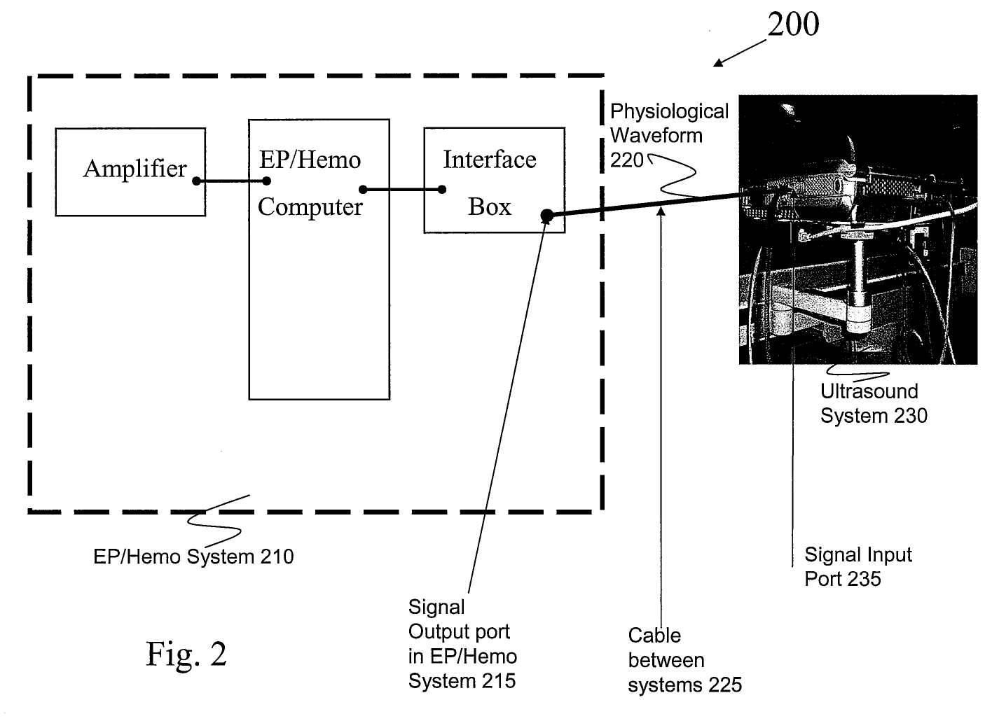

FIG. 2 illustrates a diagram of an EP/Hemo and ultrasound system 200 in accordance with an embodiment of the present invention. The system 200 includes an EP/Hemo system 210, physiological waveform data 220 and/or other data on a cable 225, and an ultrasound system 230. The ultrasound system 230 includes a signal input port 235. The EP/Hemo system 210 also includes a signal output port 215, among other components.

As described above, data, such as physiological waveform data 220, is acquired from a patient or external system via the EP/Hemo system 210. The data 220 is transmitted from the EP/Hemo system 210 via the signal output port 215. The waveform data 220 is transmitted to the ultrasound system 230 via the cable connection 225. Note that the cable connection 225 may encompass a variety of cable connections, as well as non-cable connections such as wireless, infrared, etc. The data 220 is received at the signal input port 235 of the ultrasound system 230. Similarly, data 220 may be communicated from the ultrasound system 230 to the EP/Hemo system 210 via the connection 225 and ports 235, 215.

As shown in FIG. 2, the EP/Hemo system 210 may include an amplifier for patient waveform data amplification. The system 210 may also include an EP/Hemo computer for processing and/or storage waveform and/or other data, for example. The system 210 may also include an interface for facilitating communication and/or data transfer between the system 210 and the ultrasound system 230 via the connection 225.

The waveform data 220 may be displayed and/or used in diagnosis and/or reporting at the EP/Hemo system 210 and/or ultrasound system 230, for example. The waveform data 220 may be correlated with image data from the ultrasound system 230 for processing and/or display, for example.

As discussed above in relation to FIG. 2, the EP/Hemo system 210 may include a computing device, an interface, and/or an amplifier, for example. The system 210 may also include an analog signal output and one or more single and/or multi-parameter measurement monitors for obtaining, processing and/or relaying physiological and/or other data for a patient.

In certain embodiments, a Radiology Information System (RIS) and/or other healthcare information system, such as a Picture Archiving and Communication System (PACS), etc., provides access to an ultrasound and/or other Diagnostic Imaging (DI) scanner from the RIS or other healthcare workstation, for example. Using an interface at the workstation, a user may perform a plurality of functions at the DI scanner.

A user may use the interface to automatically send report and key images to a referring physician, for example. Via the interface, a user may pull up and review previous exams, results, history and diagnosis, for example. A user may save a dose report and keep a running history in the RIS. A user may change/add a current exam order. A user may check a patient's previous exam history and review reports. In certain embodiments, a user may execute Multiple Perform Procedure Step (MPPS) commands such as start and end procedure, in progress, in transport, patient waiting location, status of reports (dictated, waiting for read, in progress), etc.

Access to a DI unit via an information system, such as a RIS, helps provide time, efficiency and quality control benefits to providers of Diagnostic Imaging services. Combining tools and information available at an information system and a diagnostic imaging system helps allow early correction of patient order entry errors and provides an opportunity for more appropriate patient care based upon patient indications while the user and/or the patient is actually at the scanner. Further, patient information may be made available to the RIS, and therefore RIS users, early in the DI services cycle for each patient, for example.

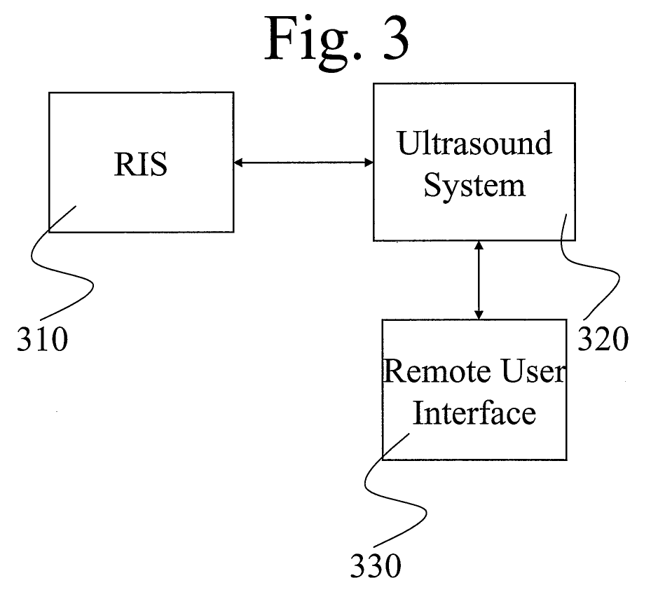

FIG. 3 illustrates a block diagram for a system 300 for interactive communication between a healthcare information system, such as a RIS, and an imaging system, such as an ultrasound system, in accordance with an embodiment of the present invention. The system 300 includes a RIS 310, an ultrasound system 320 and a remote control interface 330 connected via one or more wired and/or wireless communication connections. The RIS 310 includes patient information, order information, scanning protocol information, etc. The ultrasound system 320 includes an image acquisition scanner, for example. The remote control interface 330 may allow a user to access the ultrasound system 320, the RIS 310, etc. The remote control interface 330 may be implemented separately from and/or integrated with the RIS 310 and/or ultrasound system 320, for example. The components of the system 300 may be implemented in hardware, software and/or firmware alone and/or in various combinations, for example.

Using the interface, a user may view available information at the RIS 210 and the ultrasound system 320, for example. In certain embodiments, the user may also modify information and/or settings at the RIS 310 and/or ultrasound system 320. In certain embodiments, the interface may enable access to other information system(s) and/or imaging system(s) from the RIS 310.

From the RIS 310, a user may access a patient's imaging exam order and/or imaging exam data (e.g., image(s)). The user may review the exam order, image data and/or other patient information and make changes to correct identified errors. Then, the exam may be correctly executed at the ultrasound system scanner 320. The user may review other patient information to add in examining and/or conversing with the patient during the imaging exam. The user may also execute procedure commands and/or update patient/exam status at the ultrasound system 320 remotely via the interface 330 at the RIS 310, for example. Using the interface 330, the user may also enter notes into a patient and/or exam report stored at the RIS 310 during a scan of the patient at the ultrasound system 320.

Certain embodiments provide a mechanism to remotely operate and otherwise control the ultrasound scanner 320 and perform operations to acquire and manipulate ultrasound images. Certain embodiments provide an ability to have an operator outside a vicinity of a patient to operate the ultrasound device 320. In certain embodiments, the ultrasound system 320 may be operated by a person remotely and/or by a remote operator in conjunction with a person at the ultrasound system 320. Remote operation allows a reduction in a number of people in a procedure room, for example.

In certain embodiments, a remote user interface 330 provides an ability to have a single operator perform multiple tasks during an EP or other procedure. For example, a user may manipulate the ultrasound system 320 in conjunction to other devices used during the EP procedure such as a recording system, stimulator, etc. Such multi-tasking may reduce the personnel involved.

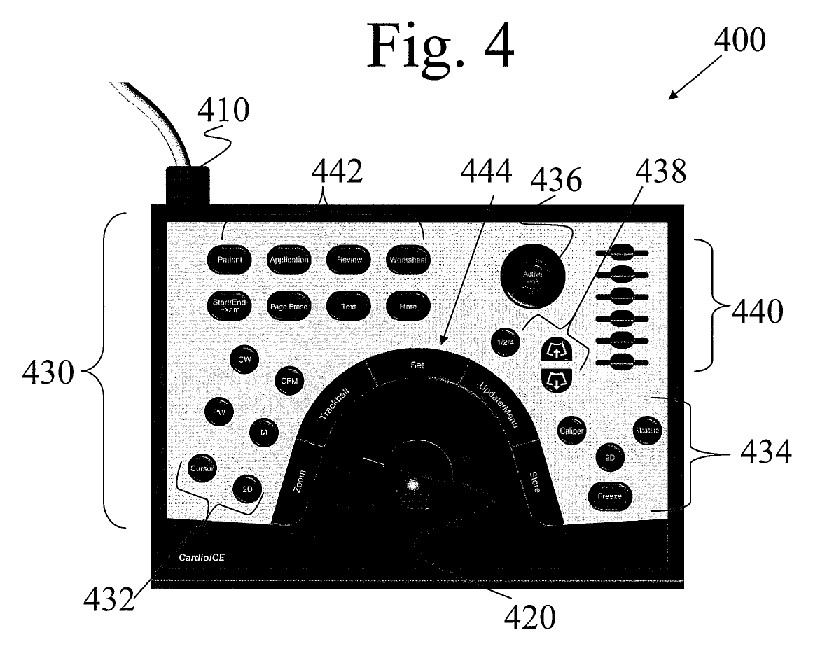

FIG. 4 illustrates a remote control 400 for an ultrasound system in accordance with an embodiment of the present invention. The remote control 400 serves as a user interface for the ultrasound device, for example. The control 400 may be wired and/or wireless connected to and/or in communication with an ultrasound device and/or other system to remotely control the ultrasound device. For example, a cable 410 may be used to connect the remote control 400 to an ultrasound system and/or intermediary computer.

In certain embodiments, the remote control 400 includes buttons, knobs, keys, etc., to control the ultrasound device. Using the remote 400, an operator may perform operations remotely rather than directly pressing keys, buttons and/or knobs on the ultrasound device. The remote control 400 may also include a trackball 420 and/or other similar device to select among options on a display screen. As shown for purposes of illustration only, one or more buttons 430, slide bars 440, etc., may be used to control ultrasound system functions. As illustrated in FIG. 4, buttons 432 may be used to select an ultrasound imaging mode, for example. One or more buttons 434 may be used to trigger operations on obtained image data, for example. One or more buttons may be used to activate imaging 436 and/or adjust scanning angle/window 438, for example. Additionally, one or more buttons 442, 444 may be mapped to one or more applications and/or functions at the ultrasound and/or other connected system.

The remote control 400 provides a mechanism to remotely operate and otherwise control the ultrasound scanner and perform operations to acquire and manipulate ultrasound images and related data, for example. Using the remote 400, an operator outside a vicinity of a patient can operate the ultrasound device. In certain embodiments, the remote 400 may be used and/or provide freedom to an operator to execute a plurality of functions at the ultrasound system and/or other device, such as recording patient data, stimulation, etc.

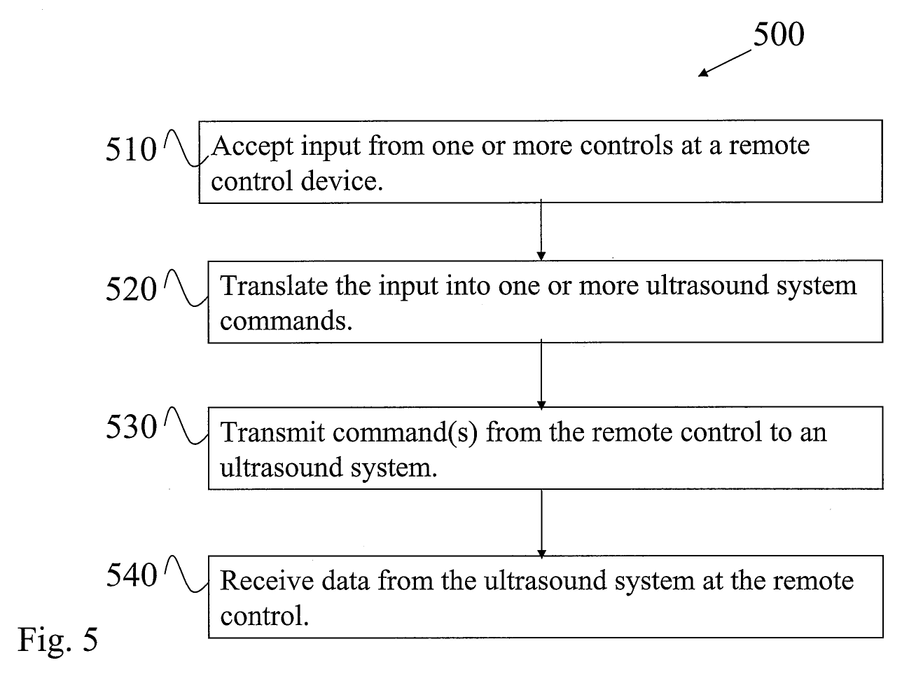

FIG. 5 illustrates a flow diagram for a method 500 for remote control of an ultrasound system in accordance with an embodiment of the present invention. At step 510, input is accepted from one or more controls at a remote control device. For example, a user manipulates one or more buttons, knobs, trackballs, slide bars, etc., on a remote control to control an ultrasound-system. At step 520, the input is translated into one or more ultrasound system commands for transmission to an ultrasound system. For example, a button push, knob turn, trackball movement, sliding bar, etc., is converted into an electronic code or signal for transmission to an ultrasound system. At step 530, the one or more commands are transmitted from the remote control to the ultrasound system for execution of the command(s) at the ultrasound system. For example, electronic signal(s) representing the command(s) are transmitted via a communication connection, such as a wireless connection or cable/wire-based connection from the remote control to the ultrasound system. At the ultrasound system, the command(s) are processed and executed. At step 540, one or more commands and/or data are transmitted from the ultrasound system to the remote control. For example, the ultrasound system may communicate with the remote control and indicate system state and/or control a remote control mode (e.g., control illumination of various buttons, etc.). In certain embodiments, an acknowledgement or feedback may be returned to the remote control upon receipt and/or execution of the command(s), for example.

In certain embodiments, the ultrasound system may be manipulated via the remote control in conjunction with one or more electrophysiologic data acquisition devices (e.g., recording system, stimulator, etc.), for example. In certain embodiments, the ultrasound system may be operated outside a vicinity of a patient using the remote control to obtain image data for the patient, for example. In certain embodiments, command(s) from the remote control allow a user to select an ultrasound imaging mode, activate imaging and/or trigger operations on obtained image data, among other things, at the ultrasound system via the remote control. In certain embodiments, the remote control may be used to access information from a healthcare information system and/or EP/Hemo system, for example. In certain embodiments, command(s) from the remote control allow a user to control a healthcare information system via the remote control, for example.

One or more of the steps of the method 500 may be implemented alone or in combination in hardware, firmware, and/or as a set of instructions in software, for example. Certain embodiments may be provided as a set of instructions residing on a computer-readable medium, such as a memory, hard disk, DVD, or ,CD, for execution on a general purpose computer or other processing device.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. An ultrasound system operable by remote control, said ultrasound system comprising:
an ultrasound scanner configured to obtain image data in at least one ultrasound imaging mode; and
a remote control configured to remotely issue commands for operation of said ultrasound scanner, said remote control separate from and in communication with said ultrasound scanner, said remote control comprising:
a plurality of controls corresponding to controls of said ultrasound scanner;
a transmitter configured to send one or more commands to said ultrasound scanner; and
a processor configured to receive input from said plurality of controls and translate said input to one or more ultrasound scanner commands for transmission to said ultrasound scanner via said transmitter.

2. The system of claim 1, wherein said transmitter comprises a cable connecting said remote control with said ultrasound scanner.

3. The system of claim 1, wherein said transmitter comprises a wireless transmitter connecting said remote control with said ultrasound scanner.

4. The system of claim 1, wherein said plurality of controls comprises a plurality of buttons, knobs and keys to control the ultrasound scanner.

5. The system of claim 1, wherein said remote control allows a user to manipulate said ultrasound scanner in conjunction with one or more electrophysiologic data acquisition devices.

6. The system of claim 1, wherein said remote control allows an operator outside a vicinity of a patient to operate said ultrasound scanner to obtain image data for said patient.

7. The system of claim 1, wherein said plurality of controls allow a user to select an ultrasound imaging mode, activate imaging and trigger operations on obtained image data at said ultrasound scanner.

8. The system of claim 1, wherein said remote control is configured to receive a command from said ultrasound scanner for control of said remote control.

9. A remote control unit configured to remotely issue commands for operation of an ultrasound system, said remote control unit separate from and in communication with said ultrasound system, said remote control unit comprising:
a plurality of controls corresponding to controls of said ultrasound system;
a transmitter configured to send one or more commands to said ultrasound system; and
a processor configured to receive input from said plurality of controls and translate said input to one or more ultrasound system commands for transmission to said ultrasound system via said transmitter.

10. The remote control unit of claim 9, wherein said transmitter comprises a cable connecting said remote control unit with said ultrasound system.

11. The remote control unit of claim 9, wherein said transmitter comprises a wireless transmitter connecting said remote control unit with said ultrasound system.

12. The remote control unit of claim 9, wherein said plurality of controls comprises a plurality of buttons, knobs and keys to control the ultrasound system.

13. The remote control unit of claim 9, wherein said remote control unit allows a user to manipulate said ultrasound system in conjunction with one or more electrophysiologic data acquisition devices.

14. The remote control unit of claim 9, wherein said remote control unit allows an operator outside a vicinity of a patient to operate said ultrasound system to obtain image data for said patient.

15. The remote control unit of claim 9, wherein said plurality of controls allow a user to select an ultrasound imaging mode, activate imaging and trigger operations on obtained image data at said ultrasound system.

16. The remote control unit of claim 9, wherein said remote control unit is configured to receive a command from said ultrasound system for control of said remote control.

17. A method for remote control of an ultrasound system, said method comprising:
accepting input from at least one of a plurality of controls at an ultrasound system remote control;
translating said input to one or more ultrasound system commands for transmission to said ultrasound system; and
transmitting said one or more commands from said remote control to an ultrasound system for execution of said one or more commands at said ultrasound system.

18. The method of claim 17, further comprising manipulating said ultrasound system via said remote control in conjunction with one or more electrophysiologic data acquisition devices.

19. The method of claim 17, wherein said one or more commands allow a user to select an ultrasound imaging mode, activate imaging and trigger operations on obtained image data at said ultrasound system via said remote control.

20. The method of claim 17, wherein said one or more commands allow a user to access a healthcare information system via said remote control.

21. The method of claim 17, further comprising receiving commands from said ultrasound system for control at said remote control.
